(19) United States
(12) Patent Application Publication
(10) Pub. No.: US 2025/0054139 A1
(43) Pub. Date: Feb. 13, 2025

(54) SYSTEM AND METHOD FOR CHARACTERIZING BIOLOGICAL TISSUE

(71) Applicant: ONCOUSTICS INC., Toronto (CA)

(72) Inventors: Adi LIGHTSTONE, Toronto (CA); Raul BLÁZQUEZ GARCÍA, Toronto (CA); Ahmed EL KAFFAS, Toronto (CA)

(21) Appl. No.: 18/721,155

(22) PCT Filed: Dec. 16, 2021
(86) PCT No.: PCT/CA2022/051851

§ 371 Date: Jun. 17, 2024

Related U.S. Application Data
(60) Provisional application No. 63/290,970, filed on Dec. 17, 2021.

(51) Int. Cl. G06T 7/00; G06V 10/82

(52) U.S. Cl. CPC G06T 7/0012; G06V 10/82; G06T 2207/10132; G06T 2207/20081; G06T 2207/20084; G06T 2207/30081; G06T 2207/30096; G06V 2201/03

(57) ABSTRACT

A system and method for characterising tissues are provided. The system comprises a processor, and a memory comprising instructions which when executed by the processor, configure the processor to perform the method. The method comprises receiving raw data corresponding to a dense two-dimensional (2D) image or signals arising from a scan of tissues within a system of interest, generating a three-dimensional (3D) data set from the dense 2D image, and inputting the 3D data set into a convolutional network having a plurality of filters. The convolutional network converting the 3D data set into a 1D array corresponding to the frequency domain of the 3D data set, and extracting features form the 1D array and classify the 1D array into a tissue pathology classification.

### CROSS-REFERENCE TO RELATED APPLICATIONS

This claims the benefit of U.S. Provisional Patent No. 63/290,970, filed Dec. 17, 2021, the entire contents of which are incorporated herein by reference.

### FIELD

The present disclosure generally relates to artificial intelligence, and in particular to a system and method for characterizing biological tissue.

### INTRODUCTION

Prostate cancer is the second most common cause of cancer death in American men. Diagnosis of prostate cancer is performed using transrectal ultrasound (TRUS)-guided biopsy. However, while many prostate cancers are hypoechoic on B-mode ultrasound, sensitivity for cancer is low. As a result, TRUS is most often used to guide 10-12 core systematic sampling, rather than identifying cancer foci for targeted sampling.

In response to the weaknesses of TRUS, MRI has been increasingly adopted based on its clear superiority over conventional TRUS. The MRI-guided biopsy approach increases detection of clinically significant prostate cancer and reduces over-detection of indolent tumors. However, MRI is expensive, not widely available, misses some cancers, and varies widely in quality of image acquisition and interpretation.

### SUMMARY

In accordance with an aspect, there is provided a system for combining multiple and variant diagnostics outputs and biomarkers to enhance diagnostics assessment and risk stratification.

In accordance with an aspect, there is provided a diagnostic system that provides an associated confidence value based on reliability and concordance of inputs (e.g. various diagnostic/biomarker inputs wherein various inputs can be combined and weighted).

In accordance with another aspect, there is provided a diagnostic system that requires only a single input to produce a diagnostics assessment.

In accordance with another aspect, there is provided a method for applying machine learning to combine disparate diagnostic inputs for enhancing confidence in a diagnostics assessment.

In accordance with another aspect, there is provided a method for applying machine learning to divergent biomarker scores or assessments for enhanced diagnostic assessment and confidence in said assessment.

In accordance with another aspect, there is a system for weighting and assessing divergent biomarker scores or assessments for enhanced diagnostic assessment and confident in said assessment.

In accordance with another aspect, there is provided a system for characterising tissues. The system comprises a processor, and a memory comprising instructions which when executed by the processor configure the processor to receive raw data corresponding to a dense two-dimensional (2D) image or signals arising from a scan of a tissue, generate a three-dimensional (3D) data set from the dense 2D image, and input the 3D data set into a convolutional network having a plurality of filters. The convolutional network is configured to convert the 3D data set into a 1D array corresponding to the frequency domain of the 3D data set, and extract features form the 1D array and classify the 1D array into a tissue pathology classification.

In accordance with another aspect, there is provided a method of characterising tissues. The method comprises receiving raw data corresponding to a dense two-dimensional (2D) image or signals arising from a scan of a tissue, generating a three-dimensional (3D) data set from the dense 2D image, and inputting the 3D data set into a convolutional network having a plurality of filters. The convolutional network converting the 3D data set into a 1D array corresponding to the frequency domain of the 3D data set, and extracting features form the 1D array and classify the 1D array into a tissue pathology classification.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

Embodiments will be described, by way of example only, with reference to the attached figures, wherein in the figures:

FIG. 1 illustrates, in a component diagram, an example of an ultrasound system, in accordance with some embodiments;

FIG. 2 shows an example of a representative B-mode image as displayed on the an imaging system indicating location of lesion and the biopsy needle trajectory, in accordance with some embodiments;

FIG. 3 illustrates an example of an RF data matrix, in accordance with some embodiments;

FIG. 4 illustrates an example of a diagram of 3D convolution network movement through the region of interest around the biopsy line, in accordance with some embodiments;

FIG. 5 illustrates, in a high-level diagram, an example of a main DL network components, in accordance with some embodiments;

FIG. 6 illustrates, experiments where a new model is trained on the same training data but with random seeding, in accordance with some embodiments;

FIG. 7 illustrates a representative image showing the PRI-MUS score and location of a large lesion in tissue based on visual assessment, in accordance with some embodiments;

FIGS. 8A and 8B illustrate, twelve images from a representative patient at different anatomical locations of the prostate based on the three-letter system, in accordance with some embodiments;

FIG. 9 displays a summarized schematic of the model 900, in accordance with some embodiments;

FIG. 10 illustrates, in a schematic overview, an example of all layers in the network, in accordance with some embodiments; and

FIG. 11 is a schematic diagram of a computing device such as a server or other computer in a device such as a vehicle.

It is understood that throughout the description and figures, like features are identified by like reference numerals.

### DETAILED DESCRIPTION

Embodiments of methods, systems, and apparatus are described through reference to the drawings. Applicant notes that the described embodiments and examples are illustrative and non-limiting. Practical implementation of the features may incorporate a combination of some or all of the aspects, and features described herein should not be taken as indications of future or existing product plans.

To address the weaknesses of TRUS and MRI, a novel micro-ultrasound (micro-US) imaging tool was recently introduced. This system operates at 29 MHz and provides greatly improved resolution compared to conventional TRUS which operates at 6-12 MHz. Ultimately, micro-US improves cancer detection and real-time targeted-biopsy. Early results indicate that micro-US may serve as a useful adjunct or a viable alternative to an MRI-based approach. To guide interpretation, the Prostate Risk Identification using Micro-US (PRI-MUS) diagnostic criteria were developed. However, interpretation of micro-US remains highly operator dependent. Thus, to optimize the benefits of micro-US, it is important to improve and standardize interpretation amongst urologists in diagnostic ultrasound.

Application of artificial intelligence techniques on TRUS data holds great potential to address current clinical challenges in image interpretation by identifying suspicious areas and guiding biopsies. Deep learning has been explored for segmenting the prostate gland in TRUS, and in some reports for prostate cancer detection. Ultrasound tissue characterization (UTC) methods have also been developed to augment TRUS by improving the detection and characterization of tissue features relevant in prostate cancer. These techniques provide quantitative measurements that relate tissue acoustic properties measured from raw ultrasound to histological micro-structural tissue properties.

In some embodiments, deep learning (DL) may be applied to directly mine the raw ultrasound data in either conventional TRUS or micro-US. For example, DL may be applied on raw micro-US data, with the goal of differentiating benign from clinically significant prostate cancer (csPCa). In some embodiments, this approach can be integrated into real-time imaging to fill a role in improving image interpretation, standardizing cancer detection and guiding prostate biopsy.

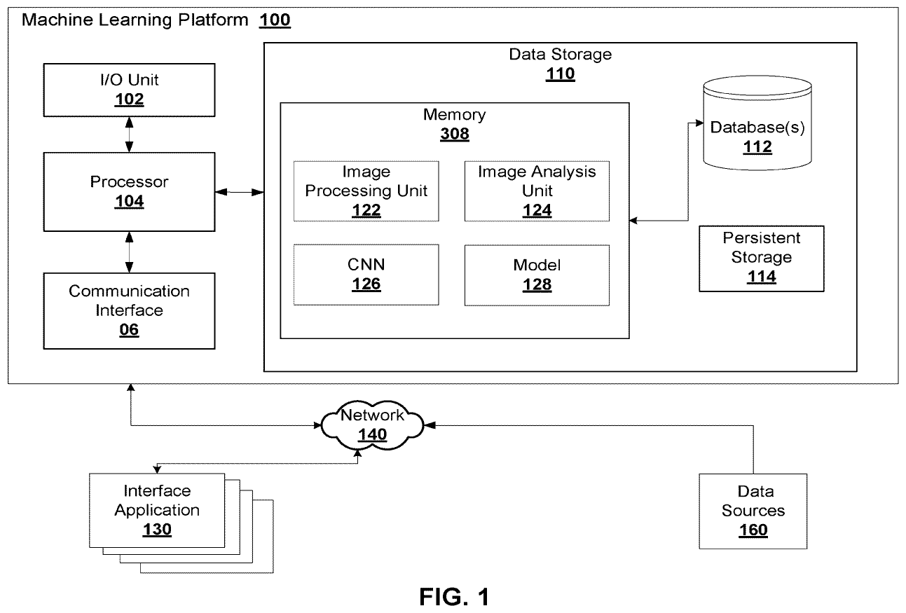

FIG. 1 illustrates, in a schematic diagram, an example of a machine learning platform 100 for characterizing tissue, in accordance with some embodiments. The platform 100 may be an electronic device connected to interface application 130 and data sources 160 via network 140. The platform 100 can implement aspects of the processes described herein.

The platform 100 may include a processor 104 and a memory 108 storing machine executable instructions to configure the processor 104 to receive a raw ultrasonic data and/or image files (e.g., from I/O unit 102 or from data sources 160). The platform 100 can include an I/O Unit 102, communication interface 106, and data storage 110. The processor 104 can execute instructions in memory 108 to implement aspects of processes described herein.

The platform 100 may be implemented on an electronic device and can include an I/O unit 102, a processor 104, a communication interface 106, and a data storage 110. The platform 100 can connect with one or more interface applications 130 or data sources 160. This connection may be over a network 140 (or multiple networks). The platform 100 may receive and transmit data from one or more of these via I/O unit 102. When data is received, I/O unit 102 transmits the data to processor 104.

The I/O unit 102 can enable the platform 100 to interconnect with one or more input devices, such as a POCUS device, keyboard, mouse, camera, touch screen and a microphone, and/or with one or more output devices such as a display screen and a speaker.

The processor 104 can be, for example, any type of general-purpose microprocessor or microcontroller, a digital signal processing (DSP) processor, an integrated circuit, a field programmable gate array (FPGA), a reconfigurable processor, or any combination thereof.

The data storage 110 can include memory 108, database(s) 112 and persistent storage 114. Memory 108 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Data storage devices 110 can include memory 108, databases 112 (e.g., graph database), and persistent storage 114.

The communication interface 106 can enable the platform 100 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g., Wi-Fi, WiMAX), SS7 signalling network, fixed line, local area network, wide area network, and others, including any combination of these.

The platform 100 can be operable to register and authenticate users (using a login, unique identifier, and password for example) prior to providing access to applications, a local network, network resources, other networks and network security devices. The platform 100 can connect to different machines or entities.

The data storage 110 may be configured to store information associated with or created by the platform 100. Storage 110 and/or persistent storage 114 may be provided using various types of storage technologies, such as solid state drives, hard disk drives, flash memory, and may be stored in various formats, such as relational databases, non-relational databases, flat files, spreadsheets, extended markup files, etc.

The memory 108 may include an image processing unit 122, an image analysis unit 124, a convolutional network 126, and a model 128.

The description herein will describe an example system and methods for the characterization of tissue(s) with reference to prostate tissue, and characterizations of prostate diagnosis. It should be understood that the present description may be adopted to other tissue (thyroid, breast, kidney, liver, bowel, pancreas, ovaries, musculoskeletal, skin and wound, or other organs, glands or tissues, etc.) characterizations for other diseases (e.g., inflammation, adenomas, steatosis or cancer, etc.). Tissue characterizations identify and differentiate tissue subtypes that naturally occur in living organisms. Tissue characterizations may facilitate diagnosis, screening, surveillance, triage and risk assessments, biopsy guidance, and surgical and guided interventions.

### Model Specifications and Training

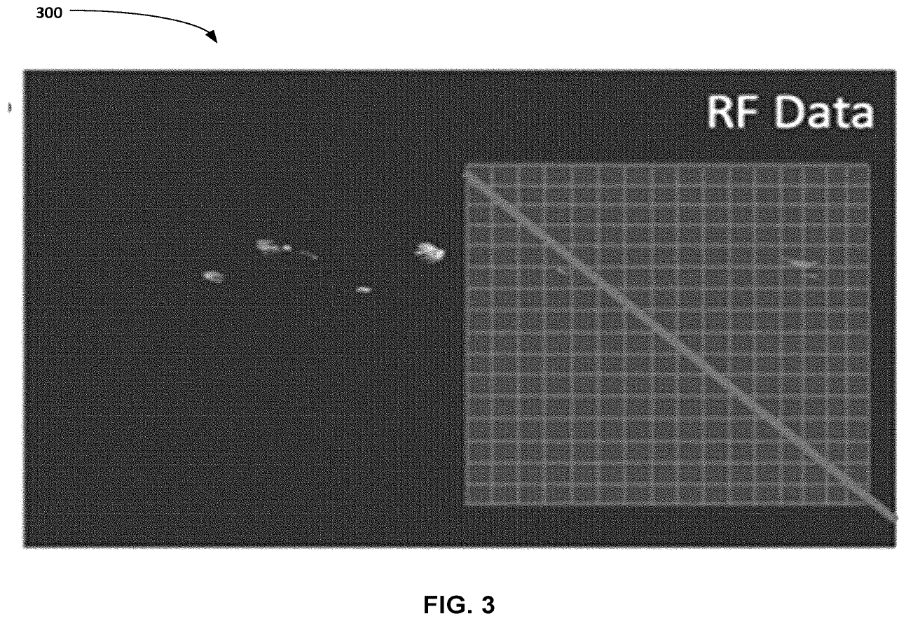

A dataset of micro-ultrasound raw data was utilized including 6530 images from 837 male patients (median age 63, IQR 57-68) undergoing prostate biopsy for suspicion of cancer. Up to 12 raw data were obtained for each patient, representing targeted biopsy locations (see representative data in FIGS. 3 to 9).

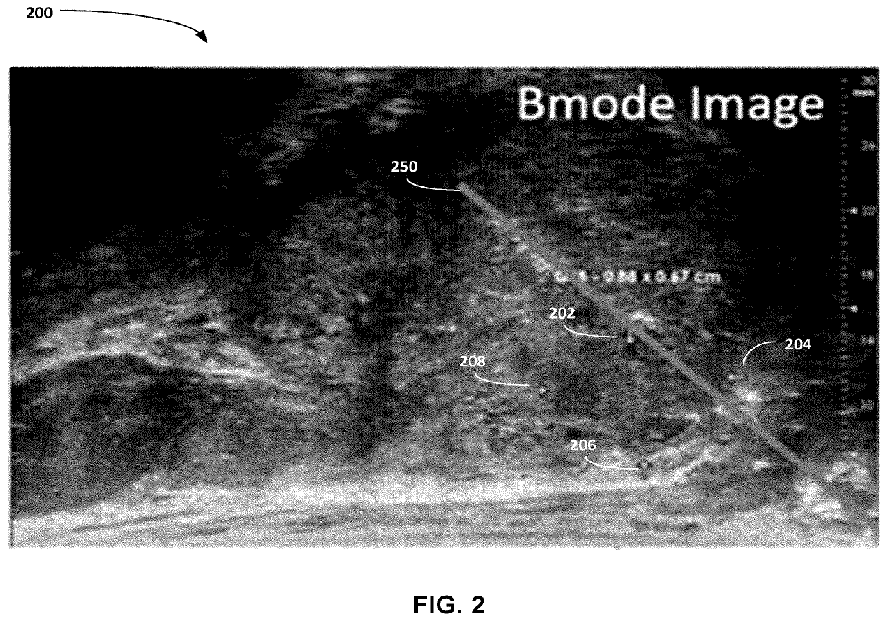

FIG. 2 shows an example of a representative B-mode image as displayed on the an imaging system indicating location of lesion (arrows 202, 204, 206, 208; Gleason score 8) and the biopsy needle trajectory 250 (e.g., the image shows in ˜28×42 mm), in accordance with some embodiments. Table 1 shows a dataset of patient demographics. Table 2 summarizes dataset characteristics of a dataset used to train and test models.

TABLE 1
Patient demographics
Patient Demographics

| Total Enrolled | 837 | |
|---|---|---|
| Age (median + IQR) | 63 | (57-68) |
| PSA (median + IQR) | 6.0 | (4.1-8.4) |
| Family History of PCa | 21.5% | |
| Positive DRE | 21.0% | |
| PC Risk Score | 44% | (38-52) |

TABLE 2
Dataset used to train and test models

| | Train/Validation | | Test (set-aside) | |
|---|---|---|---|---|
| | 5220 images | | 1310 images | |
| | (Benign 4520; G ≥ 7 700) | | (Benign 1131; G ≥ 7 179) | |
| | 369 patients | | 122 patients | |
| | Benign | ≥G7 | Benign | ≥G7 |
| PSA | 6.44 | 8.66 | 6.55 | 9.80 |
| | (std: 4.22) | (std: 5.79) | (std: 3.36) | (std: 6.35) |
| Mean | 0% | 48% | 0% | 45% |
| Pct | | (std: 30%) | | (std: 30%) |
| Cancer | | | | |
| per | | | | |
| core | | | | |

Additional details of the acquisition settings and imaging protocol are described below. As summarized in Table 2, data split included 80% of patients in the training set and 20% in a set-aside test set. The training set consisted of 5220 patient images (4520 benign vs. 700 GS≥7; 80% of the data); the testing dataset consisted of 1310 patient images (1131 benign vs. 179 GS≥7 images; 20% of data). Histopathological analysis of each biopsy sample served as the clinical standard label of benign versus clinically significant prostate cancer (csPC; Gleason score (GS)≥7). These were used as target binary labels for train and test data classification.

FIG. 3 illustrates an example of an RF data matrix 300, in accordance with some embodiments. The matrix 300 comprises 1256 samples by 512 lines, demonstrating location of the auto-selected ROI used to extract frequency power spectrums in each window. The windows allow the reduction of dense spatial information in raw data, but preserve the x-y relationship of one power spectrum to another through a 3rd frequency dimension.

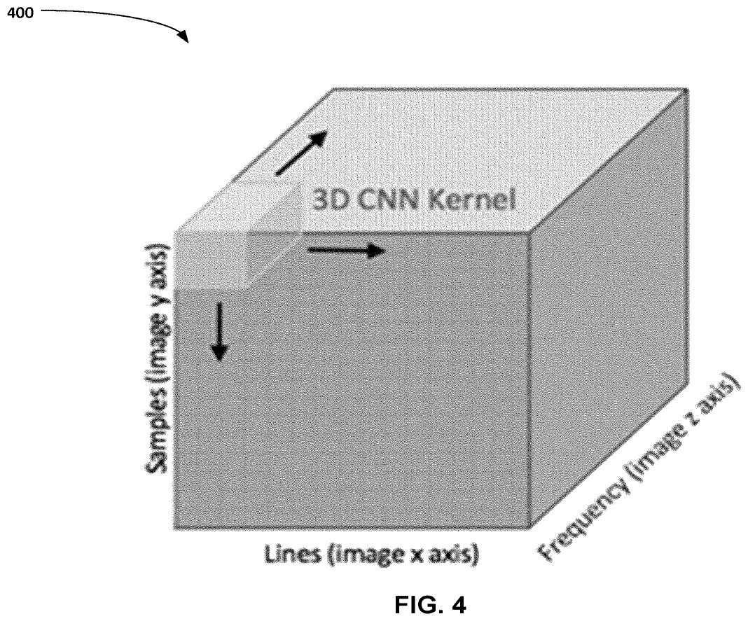

FIG. 4 illustrates an example of a diagram of 3D convolution network movement 400 through the region of interest around the biopsy line, in accordance with some embodiments. The kernel moves across spatial dimensions (samples and lines), and throughout the frequency dimension (power spectrum).

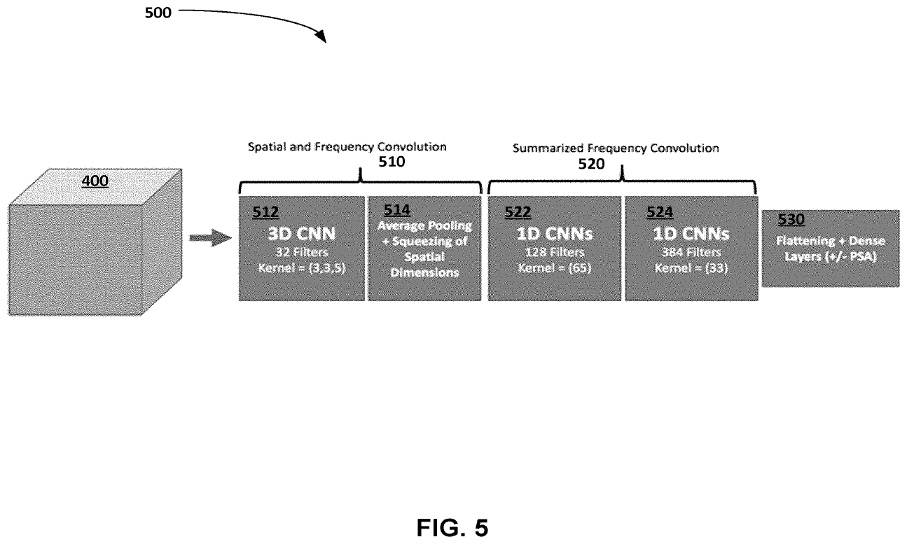

Data was processed as described below to obtain a 3D matrix (see FIG. 4) consisting of spatial information along the X-Y plane, and ultrasound power spectrums along the Z axis. This approach was used to maintain acoustic spectroscopy information (conventionally used in some UTC methods), while also maintaining the spatial information of each spectrum with respect to each other. A three-dimensional (3D) convolutional neural network (CNN) architecture was designed to first capture spatio-frequency features, and then to reduce (i.e., transform or project) the data to a one-dimensional (1D) spectrum for the final layers of the model (see FIG. 5). The model was trained on the training data (train-test split based on patient stratification). A similar network was trained with the addition of Prostate Specific Antigen (PSA) level for each patient as a feature. Training weights were used while training for class balancing and for sample balancing (based on percent cancer per sample obtained from each biopsy core, when available). No such information was used to support or augment the validation/test sets. Additional details on weights are described below. It should be understood that, in some embodiments, the 3D spatio-frequency features may be transformed or projected to a domain other than the frequency domain and/or using different types of transformations (e.g., Fast-Fourier transformation, Laplace transformation, Wavelet transformation, Z-transformations, etc.).

FIG. 5 illustrates, in a high-level diagram, an example of a main DL network components 500, in accordance with some embodiments. Starting with a 3D convolution 512, followed by reduction in spatial dimensions by pooling/squeezing 514, followed by 1D convolutions 522, 524 and dense layers 530. The PSA may be introduced on flattened data as a feature, immediately before dense layers.

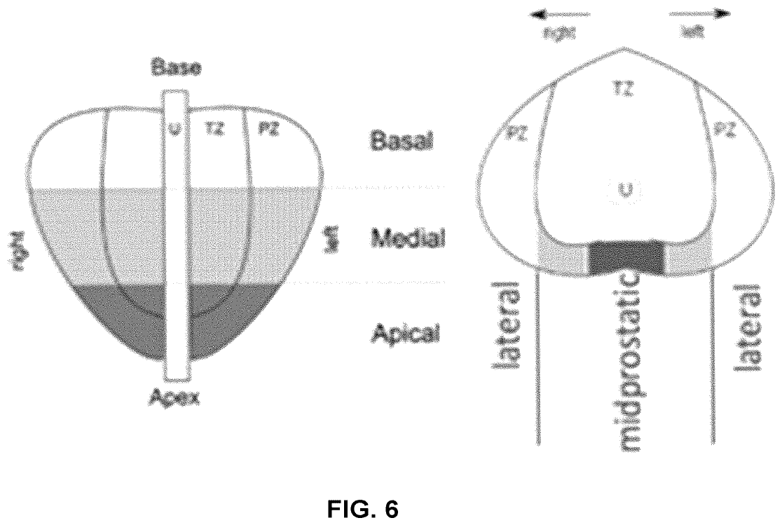

FIG. 6 illustrates, in a schematic diagram, an example of a prostate anatomy used to guide a three-letter code for a Micro-Ultrasound view, in accordance with some embodiments. The first letter may be either L (left) or R (right). The second letter may be either A (Apical), B (Basal) or M (Medial). The third letter may be either M (Midprostatic) or L (Lateral).

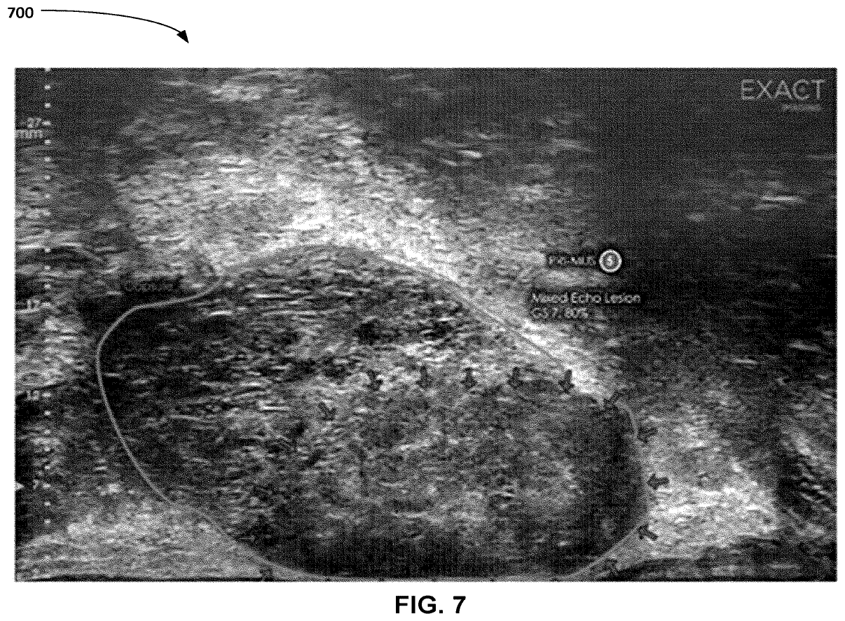

FIG. 7 illustrates a representative image 700 showing the PRI-MUS score and location of a large lesion in tissue based on visual assessment, in accordance with some embodiments.

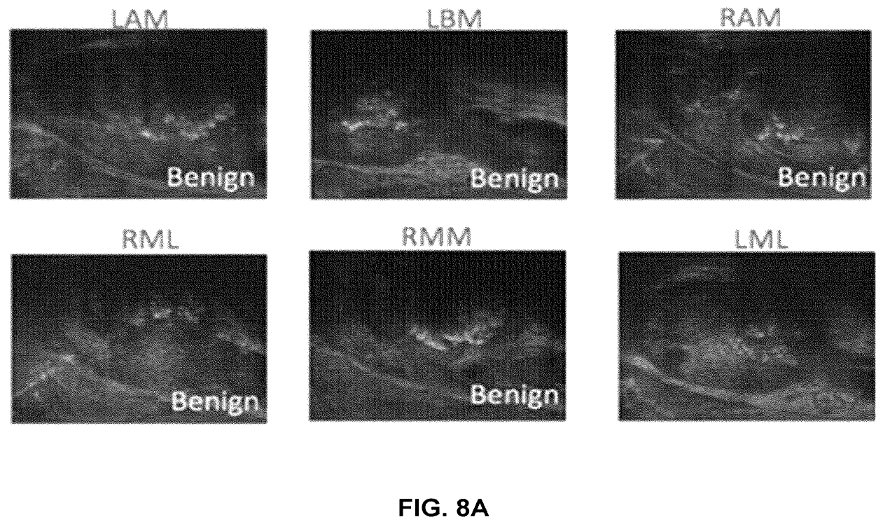

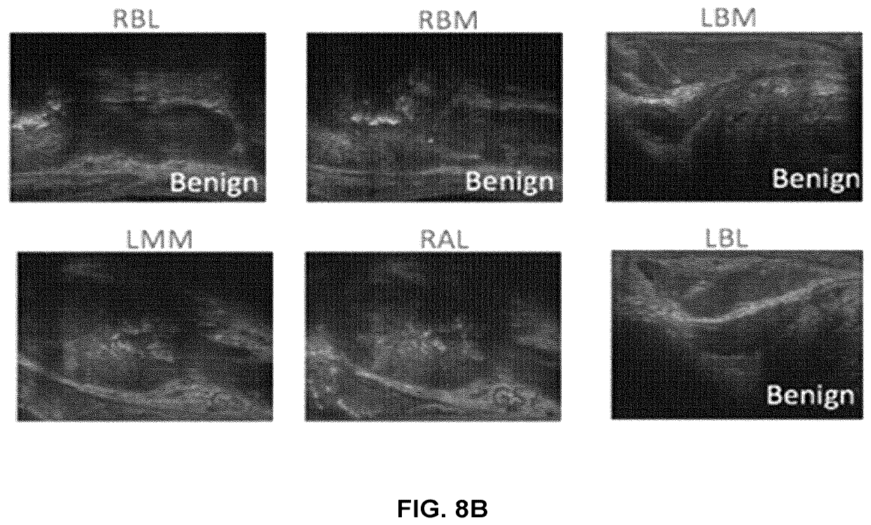

FIGS. 8A and 8B illustrate, twelve images from a representative patient at different anatomical locations of the prostate based on the three-letter system, in accordance with some embodiments. In this case, the patient has two images corresponding to biopsy-confirmed GS7. The remaining images were deemed benign from biopsy.

### Testing and Performance Evaluation

For testing the models, a set-aside data set was employed, consisting of 1310 raw image data within the region around the biopsy (see FIG. 3). All labels were confirmed by biopsy, which were read by two independent readers. Test data characteristics are summarized in Table 2.

The models have predicted csPCa in raw micro-ultrasound regions when its output (a number between 0 and 1) is above a threshold that is chosen to maximize balanced accuracy (accounts for imbalanced data by taking the arithmetic mean of sensitivity and specificity). Receiver operator curves (ROCs) that capture true positive and true negative rates at different thresholds were used to evaluate the full scale of the models. To determine the stability of the model under different initializations, ten neural networks were trained with the same set of hyperparameters and different initializations. The range between the maximum and minimum ROC curves among these realizations are the shaded regions displayed in FIG. 6. The one with best performance on the train-validation was chosen. A similar process was followed for models that include serum PSA, and the model with best performance on the train-validation ROC curves was chosen.

FIG. 6 illustrates, experiments 600 where a new model is trained on the same training data but with random seeding, in accordance with some embodiments. The experiments demonstrate stability of the model. Two left panels are the ROCs for train and test data. The right most panel is the precision-recall curve for test data. The top row is without PSA, and bottom row is with PSA. The light blue lines are 10 curves, each with random seeding. The dark blue line is the best performing model used to obtain results in Table 3 and Table 4.

### Image-Level Model Results

Table 3 displays all metrics for performance evaluation of the models on set-aside data, both with and without PSA. To summarize, a ROC-AUC of 82% was found for classifying benign vs. significant cancer (csPCa) using raw RF image data without including the PSA. This increased to 85% when including patient PSA as a model parameter. At the balanced accuracy optimized threshold, model sensitivity was 0.73 without PSA and 0.72 with PSA, indicating that PSA does not impact sensitivity. Specificity was 0.74 without PSA and 0.82 with PSA, indicating that PSA inclusion improved specificity. The F1 scores (a metric for imbalanced data) were 0.83 without PSA and 0.88 with PSA. Conversely, setting a threshold to optimize sensitivity at 0.84 produced a specificity of 0.59 (threshold 0.55) without PSA, and 0.61 (threshold of 0.68) with PSA.

TABLE 3
Image classification results

| Image | | Sensitivity | | | Balanced | F1 |
|---|---|---|---|---|---|---|
| Classification | AUC | (Recall) | Specificity | Precision | Accuracy | Score |
| Without PSA | 0.81 | 0.73 | 0.74 | 0.95 | 0.74 | 0.83 |
| (Threshold = 0.41) | | | | | | |
| With PSA | 0.85 | 0.72 | 0.82 | 0.95 | 0.81 | 0.88 |
| (Threshold = 0.48) | | | | | | |

### Patient-Level Model Results

Prediction of csPCa at a patient level was also evaluated. This is particularly clinically relevant because it could enable some men to avoid an invasive biopsy using an optimized image acquisition protocol. For this, a simplified multi-instance learning approach was used, where images were treated as the ‘instances’, and the patient was treated as the ‘bag’. Table 4 presents multi-instance patient level results. Overall, an 85% ROC-AUC was achieved in differentiating patients with benign vs. csPCa when the image-level model predicted at least three images from a patient as positive. Including the PSA improved the ROC-AUC to 91% on a patient-level.

TABLE 4
Patient-level classification results

| Patient | Positive | | Sensitivity | | | Balanced |
|---|---|---|---|---|---|---|
| Classification | Images | AUC | (Recall) | Specificity | Precision | Accuracy |
| Without PSA | 1+ | 0.77 | 0.83 | 0.64 | 0.87 | 0.73 |
| (Threshold = 0.8) | 2+ | 0.81 | 0.80 | 0.70 | 0.94 | 0.74 |
| | 3+ | 0.85 | 0.74 | 0.88 | 0.98 | 0.81 |
| With PSA | 1+ | 0.84 | 0.71 | 0.90 | 0.96 | 0.80 |
| (Threshold = 0.68) | 2+ | 0.85 | 0.71 | 0.95 | 0.99 | 0.83 |
| | 3+ | 0.91 | 0.70 | 0.99 | 0.99 | 0.85 |

Current prostate biopsy methods rely on conventional TRUS at 6-12 MHz, sometimes supplemented by MRI. However, visualization of prostate cancer on ultrasound is unreliable and MRI is expensive, not universally available, and suffers from inter-reader variability. Micro-US is a novel 29 MHz ultrasound system that improves visualization of prostate cancer. However, interpretation varies widely across users, thereby limiting the potential benefits of the new technology. In some embodiments, the power of artificial intelligence was leveraged to mine information-rich raw micro-US and ultimately improve cancer detection.

The developments demonstrate an end-to-end algorithm to classify benign and clinically significant prostate cancer using transrectal micro-ultrasound raw data. The DL-based algorithm differentiated between benign and csPCa in non-segmented micro-US raw data with an AUC of 82%. This AUC increased to 85% when patient PSA is considered as a model feature. At a patient-level, the model differentiated between benign and csPCa with an AUC of 91% (including PSA) using a simplified multi-instance learning approach.

While some cancers can be seen and characterized qualitatively in TRUS B-mode images by humans, sensitivities and specificities remain low. As such, there has been interest in using deep learning on TRUS to enhance image-guided prostate biopsy. Studies have reported median pixel-wise accuracies of up to 98% and Hausdorff distance of 3 mm to segment prostate glands, compared to manual reference standards. However, their focus was mainly on identifying the whole prostate gland for MRI fusion guidance, as opposed to identifying ‘hot zones’ linked with disease within the gland. Others have attempted to identify cancerous tissue in B-mode TRUS images, but with limited success. Challenges in developing deep learning methods to detect cancer in TRUS images include variations in B-mode acquisition parameters, operator acquisitions and system properties. These result in inconsistencies in TRUS data sets that require greater amounts of data to account for when training DL models.

Attempts to use and combine other commercially available ultrasound-based imaging modes to add dimensionality have also been explored. One study reported AUCs of up to 90% to detect csPCa when B-mode images are combined in multi-parametric models with quantitative measurements from contrast and shear wave ultrasound. While these modes are promising, they are infrequently used, in part because they are not readily available on TRUS systems in urology clinics.

UTC methods originated the use of raw ultrasound data to measure unique tissue acoustic properties related to histology. Several have explored the use of UTC methods on TRUS raw data. A technique termed quantitative ultrasound spectroscopy (QUS), has demonstrated significant diagnostic improvements to detect prostate cancer over conventional TRUS B-Mode images, with and without the use of machine learning methods. Additionally, several studies described development of a technique called temporal enhanced ultrasound (TeUS) to analyze and parameterize time-series raw radiofrequency (RF) data, with reported AUCs of up to 94% to detect malignant prostate cancer.

UTC methods have been used on micro-US data through traditional feature-based machine learning algorithms that depend on parametrization of the RF data. A ROC-AUC of up to 77% was achieved using only specific QUS parameters obtained from carefully segmented prostate tissues. Another group was able to enhance results of feature-based modeling in carefully segmented prostate tissues using a three-player minimax game method to reduce image and patch heterogeneities, resulting in a best AUC of 93%. The present disclosure builds upon these approaches in developing a featureless deep learning algorithm to differentiate benign from cancer tissues by mining directly the raw micro-US data without parameterization (i.e. UTC) or segmentation.

The present study has some findings. First, the method accurately differentiates benign from csPCa regions. The method achieved good results in detecting csPCa without the need to segment specific regions or prostate tissue for training and testing. Refining and deploying such an approach could allow for a targeted approach, where operators would be guided towards optimal regions to biopsy. This is valuable as it demonstrates its potential to aid clinicians who are not experts in micro-ultrasound interpretation in targeting cancers while minimizing the number of biopsy cores required.

Second, it was demonstrated for the first time the potential of DL in spatially mining power spectrums obtained from raw ultrasound data throughout the image space, without tissue-specific segmentation. The power spectrum is an instrumental tool in UTC-based tissue acoustic property measurements (i.e. QUS). Approaches to mine raw data with DL have been proposed, but these usually require tedious manual selection of ROIs, patching/windowing methods to deal with highly dense and rich raw RF data, and segmentation to isolate tissue types. In the approach of the present disclosure, the power spectrum was used to reduce the dense raw RF data and to facilitate entering it into a neural network, while also maintaining the spatial relationship of one window's power spectrum to another through a windowing approach. This presents a paradigm shift, where the neural network processes the whole of the image region through UTC tools, without the need to isolate specific tissues or having the user draw regions of interest.

Third, a dataset of labeled raw micro-US data was put together with consistent presets and acquisition protocols, elements used to advance the use of deep learning in raw RF data analysis using UTC methodologies. The data library consists of >6000 TRUS micro-US images obtained in >800 patients, for whom all raw ultrasound data was obtained with the same set of acquisition parameters and standards, and where all corresponding biopsy pathology results are available. This enables for the training and testing of the models on a large library of patients.

Finally, evidence was demonstrated through a simplified multi-instance learning approach that it is possible to identify patients with csPCa. This could ultimately be developed to reduce the need for biopsy.

In this study, it was demonstrated that applying a deep learning approach on raw transrectal micro-US data, without segmentation, could differentiate benign tissue from csPCa. Successful integration of this approach into real-time micro-US imaging would fill a role in improving and standardizing cancer detection and guiding prostate biopsy.

### Methods

### Experimental Design

Clinical Dataset Acquisition. Data for this work was obtained through IRB approved protocols at the local data acquisition sites (5 sites, ClinicalTrials.gov protocol NCT02079025). All patients were consented before 29 MHz micro-ultrasound data acquisition during targeted biopsy. Samples containing clinically insignificant cancer (GS6) were not analyzed.

Data Annotations and Labeling from Pathology. The position of the biopsy needle relative to the ultrasound transducer was mechanically fixed, so that a pre-specified region of the image was known to correspond to the tissue sample. A Dach biopsy specimen received standard clinical histopathology review and the full sample was classified based on the maximum and most prevalent Gleason Scores to form the Gleason Sum. The percentage of the sample containing carcinoma was also reported.

Image Processing. Patients across all sites were scanned using a consistent acquisition pre-sets for shallow imaging, and all data was saved in raw IQ format. RF data was obtained from IQ data as previously described. The raw RF frame matrix was 1256 samples (y axis) by 512 lines (x axis) and three channels (one for each focal point before blending). Data was acquired with three foci and IQ/RF data were unblended providing three RF frames for each image available. A rectangular region of interest (ROI; ˜23×32 mm) was auto-placed on each frame to intersect with the needle guiding line (see FIG. 3)—no prostate or lesion segmentation was carried out and the ROI was placed on tissues around the needle irrespective of the type of tissue within the ROI in addition to the confirmed biopsy tissue. All model training and results are obtained on data from this ROI. The ROI was then split into 18 by 18 windows (50% overlap). Each window was 2.4×3.3 mm. This was optimized to have the same number of windows along the X and Y axis. A power spectrum (PS) was obtained for each window as previously described. In brief, the PS was estimated through the square of the magnitude of the fast FFT of the Hamming-gated RF echo segment eₛ(t, xᵢ), as a function of time (t) and lateral position (xᵢ) for each scan line in the window to obtain an average power spectrum, or otherwise as expressed mathematically here:

$$PS ( f ) = {\sum }_{i = M}^{N} {\vert FFT ( Hamming ( {e}_{s} ( t , {x}_{i} ) ) \vert}^{2}$$

This results in a data matrix with 18×18 windows, each with 129 frequency points (z axis), and three channels, which were then fed into a custom network described below.

Model Design, Hyperparameter Tuning and Training. The neural network was custom designed to mine RF data for acoustic tissue signatures, and retaining the spatial information of each PS. The PS of ultrasonic RF data is commonly employed in extracting acoustic tissue properties in ultrasound tissue characterization. Here its use is expanded by leveraging CNNs to mine its unique properties spatially.

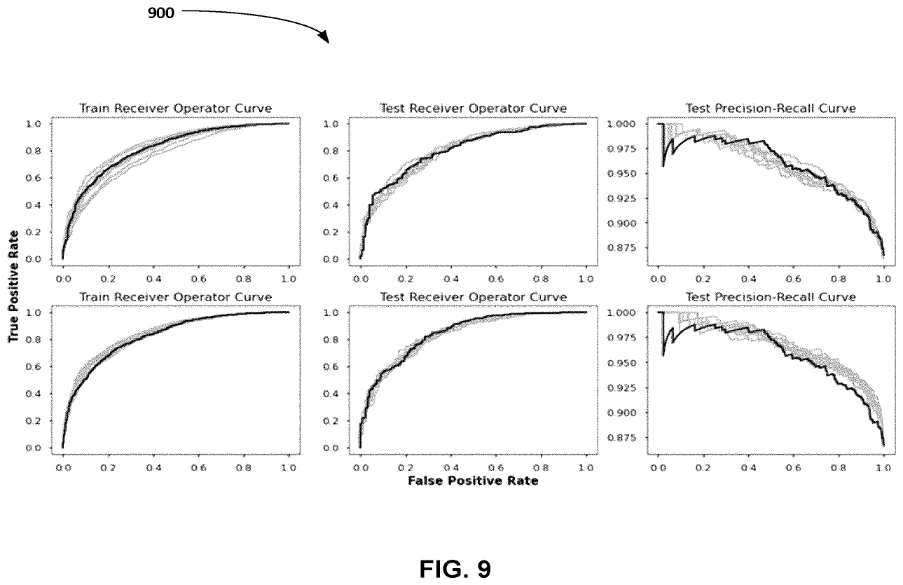

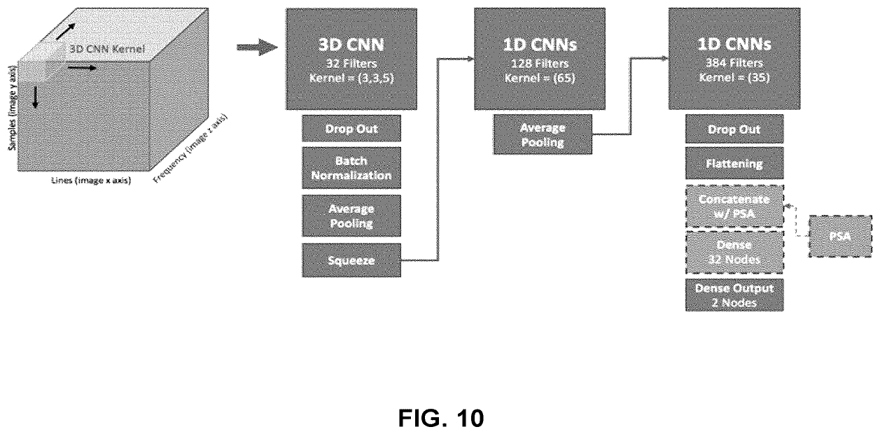

The top layers of the network includes a 3D CNN, followed by a pooling layers which aims to reduce the x-y planes by averaging. This is then followed by a series of 1D CNN layers, and finally flattening and dense layers. For models trained with PSA, the PSA was included as a feature after flattening the data. FIG. 9 displays a summarized schematic of the model 900, in accordance with some embodiments. FIG. 10 shows a more detailed schematic. FIG. 10 illustrates, in a schematic overview, an example of all layers in the network, in accordance with some embodiments. Broken lines represent components of the network that are only present when PSA is included.

The model was trained using the percent cancer from the biopsy tissue sample as a sample weight, which helped with the imbalanced data set. The percent cancer from biopsy was not considered when the model was used for predictions on set-aside test dataset.

The configuration and final architecture of the model, including the hyperparameter tuning, was obtained after >200 manual iterations of the approach using the following procedure: i) find a neural network architecture that trains along with weights, ii) check performance on the validation dataset, and iii) choose new hyperparameters and architectures. This was repeated until an ideal network, weights and hyperparameters were identified to yield suitable results.

Statistical Analysis and Reporting Metrics. The receiver operator curves and the precision-recall curve were determined to assess the model discrimination of benign from clinically significant prostate cancer (GS7). A ROC shows the true and false positive rates for a range of thresholds. The precision-recall curve shows the relationship between precision (PPV) and recall (sensitivity), calculated using binary decision thresholds for each rhythm class. For imbalanced classes, such as the test set, this plot can be more informative than the ROC plot. For the image-level analyses, the threshold that maximizes the balanced accuracy (arithmetic mean of sensitivity and specificity) was located. The F1 score was used as an additional assessment metric. The F1 score, which is the harmonic mean between precision and recall, is generally known to be robust in imbalanced data sets. From a random seed experiment (see FIG. 6), we chose the best model with the fixed threshold to compute the balanced accuracy, precision, the sensitivity (recall), the specificity, and the F1 score. Patient level analysis was carried out by taking the max of all the scores across all RFdata for that patient produced by the model.

In some embodiments, a closed loop system may be provided where the resulting classification may trigger a response or medical intervention. For example, an automated dosage of medication may be administered or ordered, a referral to/appointment with an medical expert may be automatically made, etc.

Combining Imaging with Diagnostic Methods

In some embodiments, ML may be applied to synthesize multiple Imaging and diagnostic modalities in combination to enhance sensitivity and specificity of disease diagnostics.

Discrete imaging and diagnostic methods include blood tests and different imaging modalities each of which can provide biomarkers, including molecular, histologic, radiographic and physiologic. All such biomarkers are indicators of normal biological processes, pathogenic processes, or responses to an exposure or intervention, including therapeutic interventions. The method, process and algorithms proposed combine lower specificity and sensitivity Al based diagnostics on different imaging modalities (MRI/SWE/PoCUS/Ultrasound), other diagnostic techniques (bodily fluid analysis) and patient history, along with any assessments and/or resultant AI/ML derived scores, as potential inputs and a diagnostic assessment and an associated confidence value as an output.

In some embodiments, the method/process is designed to work both with sparse and with complete data. The confidence output will depend on the spasticity of the input information. The highest confidence value will be given to automated Al diagnostics based on a complete dataset including all the information modalities. Nonetheless, the method will give high confidence values on predictions that combine 3 of the data modalities named above and the lowest confidence level for those cases with only one of the data modalities as input.

To do the diagnostic assessment, the model will use predictions on each of the imaging modalities produced by other Machine Learning algorithms particularly trained to work on those. The overall input structure will contain predictions for each of the imaging modalities, concentration of substances in bodily fluids (blood, urine, etc.) and categorical and continuous demographic and clinical history data. There may be two outputs: 1. Diagnostic assessment, 2. Confidence level of the diagnostic.

It should be noted that only one of the imaging modalities will be necessary to be available for the algorithm to produce a prediction. Nevertheless, additional inputs will boost performance.

While one of the multi-modal approaches may be lacking in sensitivity or specificity, the goal here is to strategically combine these in a way that would ultimately enhance overall sensitivity and specificity of a screening or diagnostic test. Practically, this could allow for a rapid stratification of patients, and enhance diagnostic potential of complementary systems that are already established.

In some embodiments, AI may be trained to leverage multiple imaging and diagnostic modalities in combination to offer high sensitivity and specificity diagnostics.

This AI algorithm combines lower specificity and sensitivity Al based diagnostics on different imaging modalities (MRI/SWE/PoCUS/Ultrasound), other diagnostic techniques (bodily fluid analysis) and patient history as potential inputs and a diagnostic and an associated confidence value as an output.

The algorithm is designed to work both with sparse and with complete data. The confidence output will depend on the spasticity of the input information. The highest confidence value will be given to automated Al diagnostics based on a complete dataset including all the information modalities. Nonetheless, the algorithm will give high confidence values on predictions that combine 3 of the data modalities named above and the lowest confidence level for those cases with only one of the data modalities as input.

To do the diagnostics the model will use predictions on everyone of the imaging modalities produced by other Machine Learning algorithms particularly trained to work on those. The overall input structure will contain predictions for each of the imaging modalities, concentration of substances in bodily fluids (blood, urine, etc.) and categorical and continuous demographic and clinical history data. There will be two outputs: 1. Disease diagnostic, 2. Confidence level of the diagnostic.

It should be noted that only one of the imaging modalities will be necessary to be available for the algorithm to produce a prediction. Nevertheless, any additional inputs will boost performance.

While one of the multi-modal approaches may be lacking in sensitivity or specificity, the goal here is to strategically combine these in a way that would ultimately enhance overall sensitivity and specificity of a screening or diagnostic test. Practically, this could allow for the rapid stratification of patients, and enhance diagnostic potential of complementary systems that are already established.

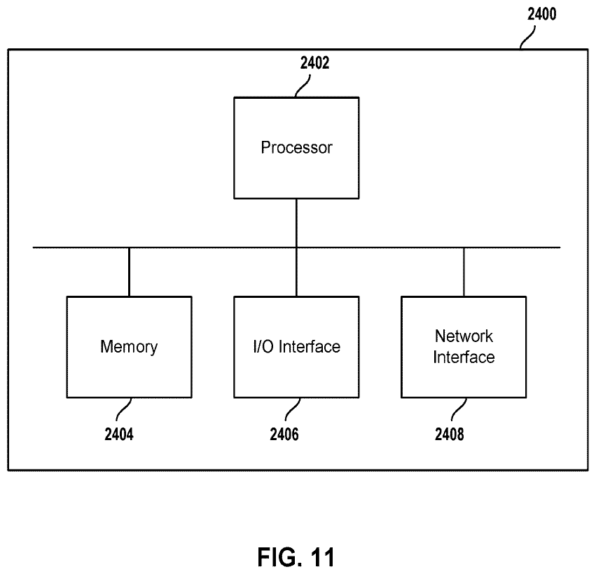

FIG. 11 is a schematic diagram of a computing device 2400 such as a server or other computer in a device such as a vehicle. As depicted, the computing device includes at least one processor 2402, memory 2404, at least one I/O interface 2406, and at least one network interface 2408.

Processor 2402 may be an Intel or AMD x86 or x64, PowerPC, ARM processor, or the like. Memory 2404 may include a suitable combination of computer memory that is located either internally or externally such as, for example, random-access memory (RAM), read-only memory (ROM), compact disc read-only memory (CDROM).

Each I/O interface 2406 enables computing device 2400 to interconnect with one or more input devices, such as a keyboard, mouse, camera, touch screen and a microphone, or with one or more output devices such as a display screen and a speaker.

Each network interface 2408 enables computing device 2400 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others.

The foregoing discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

In some embodiments, there is provided a system for combining multiple and variant diagnostics outputs and biomarkers to enhance diagnostics assessment and risk stratification.

In some embodiments, there is provided a diagnostic system that provides an associated confidence value based on reliability and concordance of inputs, wherein the inputs include a plurality of diagnostic and/or biomarker inputs.

In some embodiments, there is provided a diagnostic system that requires only a single input to produce a diagnostics assessment.

In some embodiments, there is provided a method for applying machine learning to combine disparate diagnostic inputs for enhancing confidence in a diagnostics assessment.

In some embodiments, there is provided a method for applying machine learning to divergent biomarker scores or assessments for enhanced diagnostic assessment and confidence in said assessment.

In some embodiments, there is provided a system for weighting and assessing divergent biomarker scores or assessments for enhanced diagnostic assessment and confident in said assessment.

The embodiments described herein are implemented by physical computer hardware, including computing devices, servers, receivers, transmitters, processors, memory, displays, and networks. The embodiments described herein provide useful physical machines and particularly configured computer hardware arrangements.

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification.

As can be understood, the examples described above and illustrated are intended to be exemplary only.

What is claimed is:

1. A system for characterising tissues, the system comprising:
a processor; and
a memory comprising instructions which when executed by the processor cause the processor to:
receive raw data corresponding to a dense two-dimensional (2D) image or signals arising from a scan of a tissues within a system of interest;
generate a three-dimensional (3D) data set and representation from the dense 2D image data or signals; and
input the 3D data set into a convolutional network having a plurality of filters, said convolutional network configured to:
reduce the 3D data set to a one-dimensional (1D) array corresponding to a frequency domain of the 3D data set; and
extract features from the 1D array and classify the 1D array into a tissue pathology classification based on the extracted features.

2. The system as claimed in claim 1, wherein the raw data comprises at least one of ultrasound data and/or high-resolution microscopy and/or histopathology data.

3. The system as claimed in claim 2, wherein the raw data further comprises at least one of patient demographic data, bmode data, and/or biomarker data.

4. The system as claimed in claim 1, wherein to generate the 3D data set from the dense 2D image, the processor is configured to:
discretize the 2D space through patches; and
project a frequency domain information in a third dimension.

5. The system as claimed in claim 1, wherein to generate the 3D data set from the dense 2D image, the processor is configured to:
discretize the 2D space through patches; and
transform a frequency domain information in a third dimension.

6. The system as claimed in claim 5, wherein the transformation is one of: a Fast-Fourier transformation, Laplace transformation, Wavelet transformation, Z-transformation.

7. The system as claimed in claim 1, wherein the processor is configured to at least one of:
divide the 3D data set into 3D segments;
obtain a power spectrum from raw radio frequency (RF) data corresponding to each 3D segment;
for each 3D segment:
reduce the RF data in that 3D segment into a one-dimensional (1D) array;
identify features of the tissue in that 1D array; and
populate the 1D array into an RF data matrix such that a spatial relationship of the 3D segment is maintained with respect to neighbour 3D segments;
receive a one-dimensional (1D) array representation of an image of a tissue; or
inject one or more additional clinical values in the 1D array or at other steps of a convolutional neural network.

8. The system of claim 7, wherein the one or more additional clinical values include a Prostate Specific Antigen (PSA) value and/or additional biomarkers.

9. The system as claimed in claim 1, wherein the identified features are obtained in a three dimensional (3D) matrix comprising spatial information along two planes and ultrasound power spectrums along a third plane.

10. The system as claimed in claim 9, wherein a 3D convolutional neural network is configured to capture spatio-frequency features from the ultrasound image, and reduce the features to a one dimension spectrum for final layers.

11. The system as claimed in any one of claims 1 to 10, wherein the tissue is one of several types found in: a liver, a thyroid, a breast, a kidney, a prostate, a bowel, a pancreas, an ovary, a musculoskeletal, skin and wounds, or other organs or glands.

12. A computer-implemented method of characterising tissues, the computer-implemented method comprising:
receiving raw data corresponding to a dense two-dimensional (2D) image or signals arising from a scan of tissues within a system of interest;
generating a three-dimensional (3D) data set from the dense 2D image; and
inputting the 3D data set into a convolutional network having a plurality of filters, said convolutional network:
converting the 3D data set into a 1D array corresponding to the frequency domain of the 3D data set; and
extracting features from the 1D array and classify the 1D array into a tissue pathology classification based on the extracted features.

13. The computer-implemented method as claimed in claim 12, wherein the raw data comprises ultrasound data.

14. The computer-implemented method as claimed in claim 13, wherein the raw data further comprises at least one of patient demographic data, bmode data, and/or biomarker data.

15. The computer-implemented method as claimed in claim 12, wherein generating the 3D data set from the dense 2D image comprises:
discretizing the 2D space through patches; and
projecting a frequency domain information in a third dimension.

16. The method as claimed in claim 12, comprising:
discretizing the 2D space through patches; and
transforming a frequency domain information in a third dimension.

17. The method as claimed in claim 16, wherein the transformation is one of: a Fast-Fourier transformation, Laplace transformation, Wavelet transformation, Z-transformation.

18. The computer-implemented method as claimed in claim 12, comprising at least one of:
dividing the 3D image into 3D segments;
obtaining a power spectrum from raw radio frequency (RF) data corresponding to each 3D segment;
for each 3D segment:
reducing the RF data in that 3D segment into a one-dimensional (1D) array;
identifying features of the tissue in that 1D array; and
populating the 1D array into an RF data matrix such that a spatial relationship of that 3D segment is maintained with respect to neighbour 3D segments;
receive a one-dimension (1D) array representation of an image of a tissue; or
inject one or more additional clinical values in the 1D array or at other steps of a convolutional neural network.

19. The computer-implemented method as claimed in claim 18, wherein the one or more additional clinical values include a Prostate Specific Antigen (PSA) value and/or additional biomarkers.

20. The computer-implemented method as claimed in claim 12, wherein the identified features are obtained in a three dimensional (3D) matrix comprising spatial information along two planes and ultrasound power spectrums along a third plane.

21. The computer-implemented method as claimed in claim 20, wherein a 3D convolutional neural network is configured to capture spatio-frequency features from the ultrasound image, and reduce the features to a one dimension spectrum for final layers.
